(19) United States
(12) Patent Application Publication
(10) Pub. No.: US 2005/0130301 A1
(43) Pub. Date: Jun. 16, 2005

(54) Isolation of bone marrow fraction rich in connective tissue growth components and the use thereof to promote connective tissue formation

(76) Inventors: William F. McKay, Memphis, TN (US); Jeffrey Marx, Germantown, TN (US)

Correspondence Address: Kenneth A. Gandy, Esq., Suite 3700, 111 Monument Circle, Indianapolis, IN, 46204, US

(21) Appl. No.: 10/887,275

(22) Filed: Jul. 8, 2004

Related U.S. Application Data
(60) Provisional application No. 60/485,445, filed on Jul. 9, 2003.

(51) Int. Cl. C12N 5/08

(52) U.S. Cl. 435/372

(57) ABSTRACT

A bone marrow isolate rich in one or more connective tissue growth components, methods of forming the isolate, and methods of promoting connective tissue growth using the isolate are described. A biological sample comprising bone marrow is centrifuged to separate the sample into fractions including a fraction rich in connective tissue growth components. The fraction rich in connective tissue growth components is then isolated from the separated sample. The isolate can be used directly or combined with a carrier and implanted into a patient at a tissue (e.g., bone) defect site. The biological sample can comprise bone marrow and whole blood. The isolate can be modified (e.g., by transfection with a nucleic acid encoding an osteoinductive polypeptide operably linked to a promoter) prior to application to the tissue defect site. The isolate can be made and applied to the tissue defect site in a single procedure (i.e., intraoperatively).

### REFERENCE TO RELATED APPLICATIONS

This application is a continuation of PCT/US/______ filed Jul. 1, 2004 and entitled ISOLATION OF BONE MARROW FRACTION RICH IN CONNECTIVE TISSUE GROWTH COMPONENTS AND THE USE THEREOF TO PROMOTE CONNECTIVE TISSUE FORMATION, and also claims the benefit of U.S. Patent Ser. No. 60/485,445 filed Jul. 9, 2003, each of which is hereby incorporated herein by reference in its entirety. This application is also related to U.S. patent application Ser. No. 10/116,729 filed Apr. 4, 2002, published as U.S. Patent Application Publication No. 2002/0182664 on Dec. 5, 2002, which is also incorporated herein by reference in its entirety.

### BACKGROUND

1. Technical Field

The present application relates generally to compositions and methods of promoting tissue growth and, in particular, to a bone marrow isolate rich in one or more connective tissue (e.g., bone) growth promoting components, methods of forming the isolate and methods of promoting connective tissue growth using the isolate.

2. Background of the Technology

Currently, when bone marrow is used in a bone grafting procedure, the marrow is typically aspirated from the iliac crest and placed directly on the bone graft without any secondary processing of the bone marrow. The majority of the bone marrow aspirate is blood which offers minimal benefit to facilitating bone formation. Further, there is a large content of platelets in blood that release undesirable growth factors such as PDGF (platelet derived growth factor), TGF-beta (transforming growth factor beta), and FGF (fibroblast growth factor) that have been shown under some circumstances to have an inhibitory effect on bone formation.

Accordingly, there exists a need for improved or alternative techniques for isolating components from bone marrow, particularly components which promote connective tissue formation, and using the isolated components in connective tissue repair procedures such as bone grafting and cartilage repair.

### SUMMARY OF THE INVENTION

In one embodiment, the invention provides a method for obtaining a bone marrow fraction. This method includes centrifuging a biological sample including whole blood and bone marrow to provide a separation of components of the sample based upon density. This separation provides the following fractions in decreasing order of density: (1) a fraction rich in blood cells; (2) a buffy coat fraction; (3) a platelet rich fraction; and (4) a platelet poor fraction. The buffy coat fraction is isolated alone or in combination with all or part of the platelet rich fraction, so as to form an isolate rich in connective tissue growth promoting components.

In another embodiment, the invention provides a method for treating a patient. The method includes isolating a bone marrow fraction including components that promote connective tissue formation, and implanting the bone marrow fraction into a patient at a tissue defect site. In accordance with the invention, the isolation of the bone marrow fraction is performed intraoperatively with the implantation.

In another embodiment, the invention provides a method for treating a patient that includes obtaining a sample from bone marrow of the patient, and centrifuging the sample to separate the sample into fractions based upon density, the fractions including a fraction rich in tissue promoting components. The fraction rich in tissue growth promoting components is isolated and is implanted into the patient. In accordance with the invention, the obtaining, centrifuging, and isolating steps are performed intraoperatively with the implanting step.

In another embodiment, the invention provides a method for obtaining a bone marrow fraction rich in connective tissue growth promoting components. The method includes centrifuging a biological sample comprising bone marrow to separate components of the sample into fractions based upon density, the fractions including a fraction rich in growth promoting components. The fraction rich in tissue growth promoting components is then isolated.

Additional embodiments of the invention as well as features and advantages will be apparent from the descriptions herein.

### BRIEF DESCRIPTION OF THE DRAWINGS

FIGS. 1-6 show testing results for the separation and isolation of a fraction rich connective tissue growth promoting components from biological samples comprising whole blood and bone marrow aspirate from six different donors wherein FIG. 1 shows the testing results for donor number 30500, FIG. 2 shows the testing results for donor number 30501, FIG. 3 shows the testing results for donor number 30506, FIG. 4 shows the testing results for donor number 30526, FIG. 5 shows the testing results for donor number 30527, and FIG. 6 shows the testing results for donor number 30561.

### DETAILED DESCRIPTION

For the purposes of promoting an understanding of the principles of the invention, reference will now be made to certain embodiments thereof and specific language will be used to describe the same. It will nevertheless be understood that no limitation of the scope of the invention is thereby intended, and alterations and modifications in the illustrated implants, and further applications of the principles of the invention as illustrated herein are contemplated as would normally occur to one skilled in the art to which the invention relates.

As disclosed above, the present invention provides isolates that are rich in one or more connective tissue (e.g., bone) growth promoting components derived from bone marrow, methods of forming the isolates and methods of promoting connective tissue growth using the isolates.

Whole blood includes the following components: plasma, red blood cells, white blood cells and platelets. The liquid portion of whole blood, which is referred to as plasma, is a protein-salt solution in which red and white blood cells and platelets are suspended. Plasma, which is 90 percent water, constitutes about 55 percent of the total blood volume. Plasma contains albumin (the chief protein constituent), fibrinogen (responsible, in part, for the clotting of blood), globulins (including antibodies) and other clotting proteins. Plasma serves a variety of functions, from maintaining a satisfactory blood pressure and providing volume to supplying critical proteins for blood clotting and immunity. Plasma is obtained by separating the liquid portion of blood from the cells suspended therein. Red blood cells (erythrocytes) contain hemoglobin, an iron-containing protein that carries oxygen throughout the body while giving blood its red color. The percentage of blood volume composed of red blood cells is called the “hematocrit.” White blood cells (leukocytes) are responsible for protecting the body from invasion by foreign substances such as bacteria, fungi and viruses. Several types of white blood cells exist for this purpose, such as granulocytes and macrophages which protect against infection by surrounding and destroying invading bacteria and viruses, and lymphocytes which aid in the immune defense. Platelets (thrombocytes) are small cellular components of blood that help the clotting process by sticking to the lining of blood vessels. Platelets prevent both massive blood loss resulting from trauma and blood vessel leakage that would otherwise occur.

If whole blood is collected and prevented from clotting by the addition of an appropriate anticoagulant, it can be centrifuged into its component parts. Centrifugation will result in the red blood cells, which have the highest density, packing to the most outer portion of the rotating container, while plasma, being the least dense will settle in the inner portion of the rotating container. Separating the plasma and red blood cells is a thin white or grayish layer called the buffy coat. The buffy coat layer includes the white blood cells and platelets, which together make up about 1 percent of the total blood volume.

Bone marrow is a complex tissue comprised of hematopoietic stem cells, red and white blood cells and their precursors, mesenchymal stem and progenitor cells, stromal cells and their precursors, and a group of cells including fibroblasts, reticulocytes, adipocytes, and endothelial cells which form a connective tissue network called “stroma”. Cells from the stroma morphologically regulate the differentiation of hematopoietic cells through direct interaction via cell surface proteins and the secretion of growth factors and are involved in the foundation and support of the bone structure. Studies using animal models have suggested that bone marrow contains “pre-stromal” cells which have the capacity to differentiate into cartilage, bone, and other connective tissue cells. Beresford “Osteogenic Stem Cells and the Stromal System of Bone and Marrow”, Clin. Orthop., 240:270, 1989. Recent evidence indicates that these cells, called pluripotent stromal stem cells or mesenchymal stem cells, have the ability to generate into several different types of cell lines (i.e., osteocytes, chondrocytes, adipocytes, etc.) upon activation. However, the mesenchymal stem cells are present in the tissue in very minute amounts with a wide variety of other cells (i.e., erythrocytes, platelets, neutrophils, lymphocytes, monocytes, eosinophils, basophils, adipocytes, etc.), and, in an inverse relationship with age, they are capable of differentiating into an assortment of connective tissues depending upon the influence of a number of bioactive factors.

According to one embodiment of the invention, a biological sample comprising bone marrow is centrifuged to separate the components of the sample into various fractions based on density, including a fraction rich in connective tissue growth promoting components such as mesenchymal stem cells. The fraction rich in connective tissue growth promoting components is then isolated. The resulting isolate can contain one or more connective tissue growth components at a higher concentration than present in the original sample. The resulting isolate can be applied directly to the site of a bone or other tissue defect. Alternatively, the isolate can be combined with a carrier and the resulting implant can be applied to the site of a bone or other tissue defect. In these regards, in certain embodiments of the invention, a cell-containing isolate fraction can be applied to the tissue defect site either alone or in combination with a carrier or other substance (e.g. another therapeutic substance) without any ex vivo expansion or other culturing of the isolate. In such uses, the isolate fraction can, if desired, be loaded into a suitable delivery device such as a syringe, catheter, or the like, without any such expansion or other culturing. The isolate can also be modified (e.g., by transfection with a nucleic acid encoding an osteogenic polypeptide) prior to application to the site of a bone or other tissue defect or for other uses. The isolate can consist essentially of bone marrow (e.g., bone marrow aspirate). For example, according to one embodiment of the invention, bone marrow aspirate can be the only cell-containing component of the isolate.

As well, the biological sample that is centrifuged can be free from cell culture medium materials, and in certain forms of the invention the biological sample that is centrifuged can consist essentially of tissue material (e.g. bone marrow material optionally in combination with blood or other tissue material) from a patient into which the resulting isolate fraction is to be implanted, optionally containing one or more anticoagulants.

According to a further embodiment of the invention, a biological sample comprising whole blood (e.g. peripheral blood) and bone marrow is centrifuged to separate components of the sample based on density. Separation of the sample results in formation of the following fractions in decreasing order of density: a red blood cell rich fraction; a white blood cell rich or buffy coat fraction; a platelet rich fraction and a platelet poor fraction. The buffy coat fraction, potentially along with all or part of the platelet rich fraction adjacent the buffy coat fraction, can then be isolated to form an isolate rich in connective tissue growth promoting components. The resulting isolate can contain one or more connective tissue growth components at a higher concentration than present in the original sample. Connective tissue growth components include, but are not limited to, mononuclear cells such as hematopoietic and mesenchymal stem cells. The connective tissue growth components can include, for example, connective tissue progenitor cells.

In addition to or as an alternative to the use of whole blood in a mixture with bone marrow material, a fraction of whole blood may be mixed with the bone marrow material in the formation of a biological sample to be processed by centrifugation. Illustratively, a red blood cell containing fraction or a plasma fraction of whole blood may be used in a biological sample to be processed in accordance with the present invention.

The whole blood or fraction thereof to be used in the preparation of the biological sample to be processed in accordance with the invention can, for example, be human tissue material. When being used to generate a material for implantation into a patient, the whole blood or whole blood fraction may be autologous, allogenic, or xenogenic to the patient. In allogenic situations, the whole blood or fraction may be typed and HLA matched blood relative to the patient.

The biological sample and/or isolate rich in connective tissue growth promoting components may also include an anti-coagulant. Suitable anticoagulants include, but are not limited to, heparin, sodium citrate and EDTA.

Further, the isolate rich in connective tissue growth promoting components can be combined with a solution (e.g., a sterile isotonic solution). Suitable isotonic solutions include, but are not limited to, phosphate buffered saline and tissue culture medium such as minimal essential medium.

As set forth above, a centrifuge can be used to separate a biological sample comprising bone marrow into various fractions including a fraction rich in connective tissue growth promoting components. The fraction rich in connective tissue growth promoting components can then be isolated and the resulting isolate can then be used in a bone grafting procedure. For example, the isolate can be placed onto or combined with autogenous bone graft and/or bone graft substitutes to improve their bone forming potential and fusion rate of the graft.

According to a further embodiment of the invention, a biological sample comprising bone marrow can be optimized for bone forming effectiveness by selectively isolating components from the sample that promote bone formation or by reducing the concentration of components in the sample which inhibit bone formation. According to an embodiment of the invention, this optimization can be performed in the operating room with the use of a portable centrifuge such as the Magellan™ centrifuge system which is manufactured by Medtronic, Inc. The resulting bone marrow isolate, which is rich in connective tissue growth components, can then be used directly or combined with a carrier such as autogenous bone graft or a bone graft substitute. The isolate can be formed (i.e., the biological sample comprising bone marrow can be obtained, separated into fractions and the fraction rich in connective tissue growth components isolated) and applied to a tissue defect site in a single procedure (i.e., intraoperatively). The tissue defect site can be a bone defect site.

In another embodiment of the invention, the isolate can be formed and applied to a tissue defect site in a patient in separate procedures. For example, in a first procedure, a bone marrow sample can be obtained from the patient. The bone marrow sample thus obtained can be processed in accordance with the invention to obtain an isolate rich in tissue promoting growth components. This processing can include processing in conjunction with a sample of whole blood, e.g. peripheral blood, of the patient, which can also be obtained during the first procedure. In a second procedure, the isolate obtained including the tissue growth promoting components can be implanted in the patient at a tissue defect site, such as a bone defect site.

As noted above, the biological sample from which the connective tissue growth rich fraction is isolated can comprise a mixture of blood (e.g., peripheral blood) and bone marrow (e.g., bone marrow aspirate). According to one embodiment of the invention, the sample can contain one part (by volume) of bone marrow to two parts by volume of blood (i.e., 1:2 volume ratio of bone marrow to blood). Other volume ratios of bone marrow to blood can also be used in the sample. For example, the volume ratio of bone marrow to -blood in the sample can be 1:1, 2:1, 1:3, 3:1, etc. The volume ratio of bone marrow to blood may for example be in the range of 1:100 to 100:1, more typically in the range of 1:3 to 3:1, and can be adjusted to achieve the desired processing characteristics and amount of isolate.

The bone marrow can be from any source, including for example, from spaces between trabeculae of cancellous or spongy bone, from medulary cavities of long bones, and/or from haversian canals. The bone marrow may be from a human or other mammalian source and, when the bone marrow is to be used to prepare material for implant in a patient, the bone marrow can be autologous, allogenic, or xenogenic with respect to the patient. For example, the bone marrow can be aspirated bone marrow (e.g., bone marrow aspirated from the iliac crest). The blood and bone marrow can each be taken from a patient, combined into a sample, and the connective tissue growth component rich fraction of the sample isolated (e.g., via centrifugation) and the isolate rich in connective tissue growth components applied to a tissue defect site. The procedure involving forming the isolate and applying the isolate to the defect site can be carried out during a single operation (i.e. intraoperatively).

According to further embodiments of the invention, the isolate rich in connective tissue growth components can have a platelet yield (i.e., platelet concentration in the isolate divided by platelet concentration in initial sample) that is greater than 2 times, 3 times or 4 times that of the initial sample. The isolate rich in connective tissue growth components can also have a hematocrit content of less than 50%, less than 25% or less than 12.5% by volume. According to one embodiment of the invention, the isolate rich in connective tissue growth components can have a platelet yield (i.e., platelet concentration in the isolate divided by platelet concentration in initial sample) greater than 4 times that of the initial sample and a hematocrit content of less than 12.5% by volume.

As set forth above, separation of the biological sample comprising bone marrow into various fractions including a fraction rich in connective tissue growth components can be performed using a centrifuge system. Any centrifuge system capable of separating a biological sample (e.g., a sample comprising blood) into fractions can be used. An exemplary centrifuge is the Magellan™ Autologous Platelet Separator (APS) system, manufactured by Medtronic, Inc. Centrifuge systems and methods of separating blood into various fractions are disclosed in the following U.S. patent applications: U.S. patent application Ser. No. 09/832,517, filed Apr. 9, 2001, published Feb. 21, 2002 as U.S. Patent Application Publication No. 20020022213; U.S. patent application Ser. No. 09/832,463, filed Apr. 9, 2001, published Oct. 10, 2002 as U.S. Patent Application Publication No. 20020147094; U.S. patent application Ser. No. 09/833,234, filed Apr. 9, 2001, published Dec. 27, 2001 as U.S. Patent Application Publication No. 20010055621; U.S. patent application Ser. No. 09/961,793, filed Sep. 24, 2001, published Mar. 27, 2003 as U.S. Patent Application Publication No. 20030060352; U.S. patent application Ser. No. 10/116,729, filed Apr. 4, 2002, published Dec. 5, 2002 as U.S. Patent Application Publication No. 20020182664; and U.S. patent application Ser. No. 09/833,230, fled Apr. 9, 2001, published Oct. 10, 2002 as U.S. Patent Application Publication No. 20020147098. Each of these applications is incorporated herein by reference in its entirety. The methods and systems disclosed in these applications can be used to isolate the connective tissue growth component rich fraction from a biological sample comprising bone marrow. In particular, a sample comprising blood and bone marrow can be centrifuged and the fraction corresponding to the buffy coat fraction (i.e., the second most dense fraction) and all or part of the platelet rich plasma fraction (i.e., the denser region of the plasma layer adjacent the buffy coat fraction) can be isolated using an apparatus and method as disclosed in the aforementioned applications. According to an embodiment of the invention, the apparatus can comprise a sensor assembly which can be used to identify the interfaces between separated fractions of the sample based on changes in fluid density. For example, the interface between the region rich in red blood cells and the buffy coat fraction or platelet rich plasma fraction and the interface between the platelet rich plasma fraction and a platelet poor plasma fraction can be identified using a sensor assembly as set forth in the aforementioned applications. Knowledge of the location of the interfaces between the separated fractions of the sample can be used to isolate the desired fraction from the sample.

The connective tissue growth component rich fraction which is isolated from the biological sample can comprise the buffy coat fraction (i.e., the second most dense fraction) and all or part of the platelet rich plasma fraction (i.e., the denser region of the plasma layer adjacent the buffy coat fraction) resulting from the separation of the sample comprising blood and bone marrow. According to a further embodiment of the invention, the isolate can comprise up to 50% by volume of the sample. For example, the isolate can comprise up to 40%, 30%, or 20% by volume of the sample. According to a preferred embodiment of the invention, the connective tissue growth component rich fraction which is isolated from the biological sample can comprise from 5 to 17 percent by volume of the original sample. For example, in a 60 cc sample, the isolate can have a volume of from 3 to 10 cc. According to a further embodiment, the isolate can comprise approximately 10% by volume of the original sample (e.g., 6 cc of isolate for a 60 cc sample). Although a 60 cc sample volume is disclosed above, larger or smaller volume biological samples can also be used. For example, the volume of the biological sample can be chosen based on the amount of blood or bone marrow available and/or on the amount of isolate required for a given procedure. For example, the biological sample can have a volume of up to 100 cc, 75 cc, 50 cc, or 25 cc.

Centrifugation of the sample is conducted for a time and at a rate of rotation sufficient to achieve the desired degree of separation. For example, centrifugation can be conducted for approximately 60 seconds to 10 minutes at a rate of rotation between 0 and 5,000 rpm. According to one embodiment of the invention, centrifugation is conducted for 17 to 20 minutes. It will be understood by those of skill in the art that faster speeds of rotation will generally separate the components of the biological sample in a shorter period of time. Generally, it will be desirable to achieve the separation over a period of time of about 60 minutes or less. Further, when a bone marrow material is harvested from a patient to develop a fraction for re-implantation, the centrifugation of the biological sample including bone marrow is desirably conducted soon after harvest of the bone marrow, for example within about 2 hours and desirably within about 1 hour. As well, the re-implantation of such an isolate fraction in accordance with the invention can take place soon after obtaining the isolate fraction, for example within about 2 hours, and desirably within about 1 hour. In still further embodiments of the invention, the harvest of the bone marrow fraction, the centrifugation to obtain the isolate fraction, and the implantation of the isolate fraction can all occur on the same day, e.g. in the course of no more than about 3 hours.

As disclosed above, in one mode of use, an isolated fraction of the invention can be used for implantation in a patient. As well, isolates of the invention can be used as a source of components which may be further purified, e.g. in the recovery of isolated cells from the isolate fraction, and/or in diagnostics or research pertaining to the components therein, for example in research pertaining to cells contained in an isolate fraction.

The implantation of isolates in accordance with the invention can be made in order to treat a broad variety of tissue defects for maladies. Illustrative tissue defects that may be treated include defects in bone, neural, muscle, tendon, dermis, and marrow stroma tissues. Illustrative bone tissues that may be repaired include those of the sternum, cranium, long bones, spinal elements such as vertebra, and generally in the repair of tissue damage relating to bone cysts. Illustrative neural tissues that may be repaired include both central and peripheral nervous tissue. Cartilaginous tissue can also be treated with implants in accordance with the invention, including treatments for joint repair, in providing therapy for osteoporosis, or in the repair of tendons and ligaments in general. Implants in the treatment of muscle tissue may be made in either cardiovascular or skeletal muscle. Implants of the invention can also be used within the spinal disc space in the repair or supplementation of disc nucleus tissue, and in implants for dental applications, for example involving bone and/or gingival tissue. In each of these or other treatments, isolates of the invention can be introduced in combination with proteins or other therapeutic substances, genes, or other beneficial materials.

In the repair of bone tissue, the isolate of the invention can optionally be combined with at least one bioactive factor that induces or accelerates the differentiation of progenitor or stem cells into the osteogenic lineage. The isolate can be contacted with the bioactive agent ex-vivo, or injected into the defect site before, during, or after the implantation of the isolate. The bioactive agent can be a member of the TGF-ss superfamily that includes various tissue growth factors, including bone morphogenic proteins such as BMP-2, BMP-3, BMP-4, BMP-6, and BMP-7.

In the repair of cartilaginous tissue, isolates of the invention may be implanted to treat shallow cartilage chondral defects or full thickness cartilage defects, to treat patellar or spinal disc cartilage, or to regenerate articular joint cartilage, e.g. in patients with osteoporosis. Joints that may be treated with isolates of the invention include, but are not limited to, knee joints, hip joints, shoulder joints, elbow joints, ankle joints, tarsal and metatarsal joints, wrist joints, spinal joints, carpal and metacarpal joints, and the temporal mandibular joint.

According to a further embodiment of the invention, the connective tissue growth component rich isolate can be modified prior to implantation. For example, cells (e.g., mesenchymal stem cells) in the connective tissue growth component rich isolate can be modified using appropriate genes and/or proteins to direct a lineage specific expansion and/or differentiation or a multi-lineage expansion or differentiation.

According to an embodiment of the invention, cells (e.g., mesenchymal stem cells) in the connective tissue growth component rich factor can be transfected with a nucleic acid comprising a nucleotide sequence which encodes an osteoinductive protein or polypeptide. Exemplary osteoinductive proteins which can be encoded by the nucleotide sequence include, but are not limited to, a BMP, an LMP or a SMAD protein or an active (i.e., an osteoinductive) portion thereof. The nucleotide sequence which encodes the osteoinductive protein or polypeptide can be operably linked to a promoter. For example, the nucleotide sequence can be in a vector such as an expression vector (e.g., an adenovirus).

Nucleic acids comprising nucleotide sequences encoding LIM mineralization proteins (LMPs) and vectors and techniques for transfecting cells with nucleic acids comprising nucleotide sequences encoding LIM mineralization proteins are disclosed in the following U.S. patent applications: U.S. patent application Ser. No. 09/124,238, filed Jul. 29, 1998, now U.S. Pat. No. 6,300,127; U.S. patent application Ser. No. 09/959,578, filed Apr. 28, 2000, pending; U.S. patent application Ser. No. 10/292,951, filed Nov. 13, 2002, published Sep. 25, 2003 as U.S. Patent Application Publication No. 20030180266; and U.S. patent application Ser. No. 10/382,844, filed on Mar. 7, 2003, published Dec. 4, 2003 as U.S. Patent Application Publication No. 20030225021. Each of these applications is incorporated by reference herein in its entirety. Any of the materials and techniques disclosed in these applications can be used to modify cells in the connective tissue growth component rich factor.

The osteoinductive polypeptide encoded by the nucleic acid can be an active (i.e., osteoinductive) portion of a human LIM mineralization protein (e.g., hLMP-1 or hLMP-3). For example, the osteoinductive polypeptide can comprise at least “n” consecutive amino acids from the sequence of hLMP-1 or hLMP-3 wherein n is 5, 10, 15 or 20.

According to a further embodiment of the invention, the osteoinductive polypeptide can be an osteoinductive portion of hLMP-1 or hLMP-3 which comprises at least “n” consecutive amino acids from the amino acid sequence:

(SEQ ID NO: 1)

ASAPAADPPRYTFAFSVSLNKTARPFGAPPPADSAPQQNG

or at least “n” consecutive amino acids from the amino acid sequence:

(SEQ ID NO: 2)

ASAPAADPPRYTFAPSVSLNKTARPFGAPPPADSAPQQN

wherein n is 5, 10, 15 or 20. According to a further embodiment of the invention, the osteoinductive polypeptide can be an osteoinductive portion of hLMP-1 or hLMP-3 which comprises at least “n” consecutive amino acids from the amino acid sequence:

| | |
|---|---|
| P P P A D S A P Q | (SEQ ID NO: 3) |

wherein n is 4, 5, 6, 7 or 8. According to a further embodiment of the invention, the osteoinductive polypeptide can be an osteoinductive portion of hLMP-1 or hLMP-3 which comprises the sequence:

| | |
|---|---|
| P P P A D. | (SEQ ID NO: 4) |

The osteoinductive polypeptide (e.g., the osteoinductive portion of the hLMP-1 or hLMP-3 protein) can comprise up to 15 amino acid residues. According to further embodiments of the invention, the osteoinductive polypeptide (e.g., the osteoinductive portion of the hLMP-1 or hLMP-3 protein) can comprise up to 20, 25, 30, 35, 40, 45 or 50 amino acid residues.

The osteoinductive polypeptide can be a synthetic polypeptide. For example, the osteoinductive polypeptide can be a synthetic polypeptide having a sequence corresponding to an osteoinductive portion of hLMP-1 or hLMP-3.

The isolate rich in connective tissue growth promoting components can also be modified with a conjugate of a protein transduction domain (PTD) and an osteoinductive protein or a nucleic acid encoding an osteoinductive protein. For example, cells (e.g., mesenchymal stem cells) in the connective tissue growth component rich factor can be contacted with a conjugate of a protein transduction domain (PTD) and an osteoinductive polypeptide or a nucleic acid encoding an osteoinductive polypeptide. The osteoinductive polypeptide can be a BMP, an LMP, a SMAD protein or an active (i.e., osteoinductive) portion of an osteoinductive protein. Conjugates of PTDs and osteoinductive proteins are disclosed in Provisional U.S. Patent Application Ser. No. 60/456,551, filed Mar. 24, 2003 which is incorporated by reference herein in its entirety. Any of the conjugates and techniques disclosed in that application can be used to modify cells in the connective tissue growth component rich factor. Conjugates of a PTD and an active (i.e., osteoinductive) portion of a human LIM mineralization protein (e.g., hLMP-1 or hLMP-3) as set forth above can also be used to modify cells in the connective tissue growth rich component rich isolate.

Cells (e.g., mesenchymal stem cells) in the connective tissue growth component rich isolate can also be contacted with an osteoinductive polypeptide. For example, the isolate can be combined with an osteoinductive protein (e.g., BMP-2). The modified isolate can then be placed on a carrier and implanted into a patient.

In this regard, carriers that may be used with isolate materials of the invention can be a dimensionally-stable or non-dimensionally-stable (e.g. paste or putty) carrier. The carrier can, for example, be a resorbable porous matrix. In this regard, the resorbable porous matrix is collagenous in certain embodiments. A wide variety of collagen materials are suitable for the resorbable matrix. Naturally occurring collagens may be subclassified into several different types depending on their amino acid sequence, carbohydrate content and presence or absence of disulfide cross-links. Types I and III collagen are two of the most common subtypes of collagen. Type I collagen is present in skin, tendon and bone whereas Type III collagen is found primarily in skin. The collagen in the matrix may be obtained from skin, bone, tendon, or cartilage and purified by methods known in the art. Alternatively, the collagen may be purchased commercially. The porous matrix composition desirably includes Type I bovine collagen.

The collagen of a carrier matrix can further be atelopeptide collagen and/or telopeptide collagen. Moreover, non-fibrillar and/or fibrillar collagen may be used. Non-fibrillar collagen is collagen that has been solubilized and has not been reconstituted into its native fibrillar form.

Suitable resorbable carrier matrix materials may also be formed of other organic materials such as natural or synthetic polymeric materials, in addition to or as an alternative to collagen. For example, the resorbable carrier may comprise gelatin (e.g. foamed gelatin), or resorbable synthetic polymers such as polylactic acid polymers, polyglycolic acid polymers, or co-polymers thereof. Other natural and synthetic polymers are also known for the formation of biocompatible resorbable matrix materials, and can be used in the invention.

The carrier may also be or include a natural and/or synthetic mineral component. For example, the mineral component can be provided by a particulate mineral material, including either powder form or larger particulate mineral materials. In certain embodiments, the particulate mineral component is effective in providing a scaffold for bone ingrowth as the resorbable matrix material is resorbed. The mineral material may for example be bone, especially cortical bone, or a synthetic bioceramic such as a biocompatible calcium phosphate ceramic. Illustrative ceramics include tricalcium phosphate, hydroxyapatite, and biphasic calcium phosphate. These mineral components may be purchased commercially or obtained or synthesized by methods known in the art.

As noted above, biphasic calcium phosphate can be used to provide a mineral-containing carrier in the invention. Desirably, such biphasic calcium phosphate will have a tricalcium phosphate:hydroxyapatite weight ratio of about 50:50 to about 95:5, more preferably about 70:30 to about 95:5, even more preferably about 80:20 to about 90:10, and most preferably about 85:15.

The carrier can include an amount of mineral that will provide a scaffold effective to remain in a patient for a period of time sufficient for the formation of osteoid in the void for which bone growth is desired. Typically, this period of time will be about 8 to about 12 weeks, although longer or shorter periods may also occur in particular situations. The minimum level of mineral that must be present in the carrier for these purposes is also dependent on the level of activity of the tissue growth promoting components in the isolate and whether other substances such as BMP or other osteogenic proteins are incorporated into the carrier in combination with the tissue growth promoting components of the isolate.

In certain forms of the invention, the carrier may include a particulate mineral component embedded in a porous organic matrix formed with a material such as collagen, gelatin or a resorbable synthetic polymer. In this regard, the particulate mineral:resorbable porous matrix weight ratio of the first implant material may be at least about 4:1, more typically at least about 10:1. In highly mineralized carriers, the particulate mineral will constitute at least 95% by weight of the first implant material. For example, carrier materials may be provided comprising about 97% to about 99% by weight particulate mineral and about 1% to about 3% of the collagen or other matrix forming material. Moreover, the mineral component may for example have an average particle size of at least about 0.5 mm, more preferably about 0.5 mm to about 5 mm, and most preferably about 1 mm to about 3 mm.

Carriers used in combination with the isolate may be non-dimensionally-stable, for example as in flowable or malleable substances such as pastes or putties. Illustratively, the carrier may include a biologically resorbable, non-dimensionally-stable material having properties allowing its implantation and retention at a tissue defect site. Such carriers can include resorbable organic materials such as macromolecules from biological or synthetic sources, for example gelatin, hyaluronic acid carboxymethyl cellulose, collagen, peptides, glycosaminoglycans, proteoglycans, and the like. Such materials can be used with or without an incorporated particulate mineral component as described hereinabove. In certain forms, the resorbable carrier can be formulated into the composition such that the composition is flowable at temperatures above the body temperature of a patient into which the material is to be implanted, but transitions to be relatively non-flowable at or slightly above such body temperature. The resorbable carrier may be formulated into the implanted composition so the flowable state is a liquid or a flowable gel, and the non-flowable state is a stable gel or solid. In certain embodiments of the invention, the resorbable carrier can include gelatin, and/or can incorporate a particulate mineral in an amount that constitutes about 20% to about 80% by volume of the carrier composition, more typically about 40% to about 80% by volume.

In certain forms of the invention, the carrier can be an osteoconductive matrix providing biologically inert surfaces which are receptive to the growth of new host bone. For example, the carrier can be a collagen sponge or another dimensionally-stable or non-dimensionally stable carrier as described above having these characteristics.

The carrier can comprise growth factors which can modulate the growth or differentiation of other cells. Growth factors which can be used include, but are not limited to, bone morphogenic proteins, sMAD proteins, and LIM mineralization proteins. Demineralized bone matrix can also be included in the carrier. For example, powders or granules of demineralized bone matrix can be incorporated into the carrier.

The isolate can also be combined with allograft and/or autograft bone. For example, the isolate can be combined with allograft and/or autograft bone and the resulting implant can then be implanted into a host. As well, before or after implantation, an isolate of the invention can be combined with one or more platelet activating agents, for example thrombin, to activate any platelets contained in the isolate, and/or with other substances relating to the blood clotting cascade such as fibrinogen.

The isolate or an implant comprising the isolate can enhance or accelerate the growth of new bone tissue by one or more mechanisms such as osteogenesis, osteoconduction and or osteoinduction. For example, the isolate or an implant comprising the isolate can have osteoinductive properties when implanted into a host. Thus, the isolate or implant comprising the isolate can recruit cells from the host which have the potential for repairing bone tissue.

The isolate rich in connective tissue growth components or an implant comprising the isolate can be used in bone repair. For example, the isolate or an implant comprising the isolate can be applied at a bone repair site, e.g., one resulting from injury, defect brought about during the course of surgery, infection, malignancy or developmental malformation. The isolate or an implant comprising the isolate can be used in a wide variety of orthopedic, periodontal, neurosurgical and oral and maxillofacial surgical procedures including, but not limited to: the repair of simple and compound fractures and non-unions; external and internal fixations; joint reconstructions such as arthrodesis; general arthroplasty; cup arthroplasty of the hip; femoral and humeral head replacement; femoral head surface replacement and total joint replacement; repairs of the vertebral column including spinal fusion and internal fixation; tumor surgery, e.g., deficit filing; discectomy; laminectomy; excision of spinal cord tumors; anterior cervical and thoracic operations; repairs of spinal injuries; scoliosis, lordosis and kyphosis treatments; intermaxillary fixation of fractures; mentoplasty; temporomandibular joint replacement; alveolar ridge augmentation and reconstruction; inlay osteoimplants; implant placement and revision; sinus lifts; cosmetic enhancement; etc. Specific bones which can be repaired or replaced with the isolate or implant comprising the isolate include, but are not limited to: the ethmoid; frontal; nasal; occipital; parietal; temporal; mandible; maxilla; zygomatic; cervical vertebra; thoracic vertebra; lumbar vertebra; sacrum; rib; sternum; clavicle; scapula; humerus; radius; ulna; carpal bones; metacarpal bones; phalanges; ilium; ischium; pubis; femur; tibia; fibula; patella; calcaneus; tarsal and metatarsal bones.

The isolate rich in connective tissue growth components or an implant comprising the isolate can also be used in cartilage repair. For example, the isolate or an implant comprising the isolate can be applied at a cartilage defect site. For example, the isolate can be used at the site of an articular cartilage defect.

The isolate rich in connective tissue growth components or an implant comprising the isolate can also be used in soft tissue repair.

The bone marrow can be aspirated bone marrow. The bone marrow can be autologous bone marrow aspirated from the patient being treated for a tissue defect. The bone marrow can be obtained using known techniques. According to an embodiment of the invention, the bone marrow can be aspirated (e.g., from the iliac crest) using Jamshedi needles.

The methods described herein for isolating a fraction rich in connective tissue growth promoting components offer numerous advantages. First, the methods do not require the use of separation media such as density gradient media, although it will be understood that in certain embodiments of the invention, the use of such separation media will be encompassed. These separation media are not approved for introduction into humans. Therefore, when separation media that cannot be introduced into the patient are employed, a series of washing steps are required to eliminate the separation media from the isolated cell populations. The preferred methods disclosed herein can be used to isolate the desired cells without the use of a separation media and therefore do not require separate washing steps. Accordingly, isolates of the invention to be implanted can be loaded into delivery devices, such as syringes, catheters, and the like, without any intervening washing step. The preferred methods described herein also allow for the intraoperative isolation and use of the isolate for tissue repair. Further, the preferred methods described herein allow for the use of relatively small sample sizes (e.g., 60 cc or less).

For the purpose of promoting a further understanding of the invention, the following Experimentals are provided. It will be understood that these Experimentals are illustrative and not limiting of the invention.

Experimental 1

The following non-limiting examples are intended to illustrate methods of forming an isolate rich in connective tissue growth promoting components from a biological sample comprising whole blood and bone marrow.

Biological samples comprising mixtures of 20 mL anticoagulated bone marrow and 40 mL anticoagulated blood were processed using the Magellan™ APS system. The fraction rich in connective tissue growth promoting components from each run was then isolated. The resulting isolate was then evaluated for platelet yield (i.e., platelet concentration in the isolate divided by the platelet concentration in the initial sample) and for hematocrit content. For each run, the isolate had a volume of approximately 6 cc and included the buffy coat fraction and portions of the adjacent platelet rich fraction of the sample.

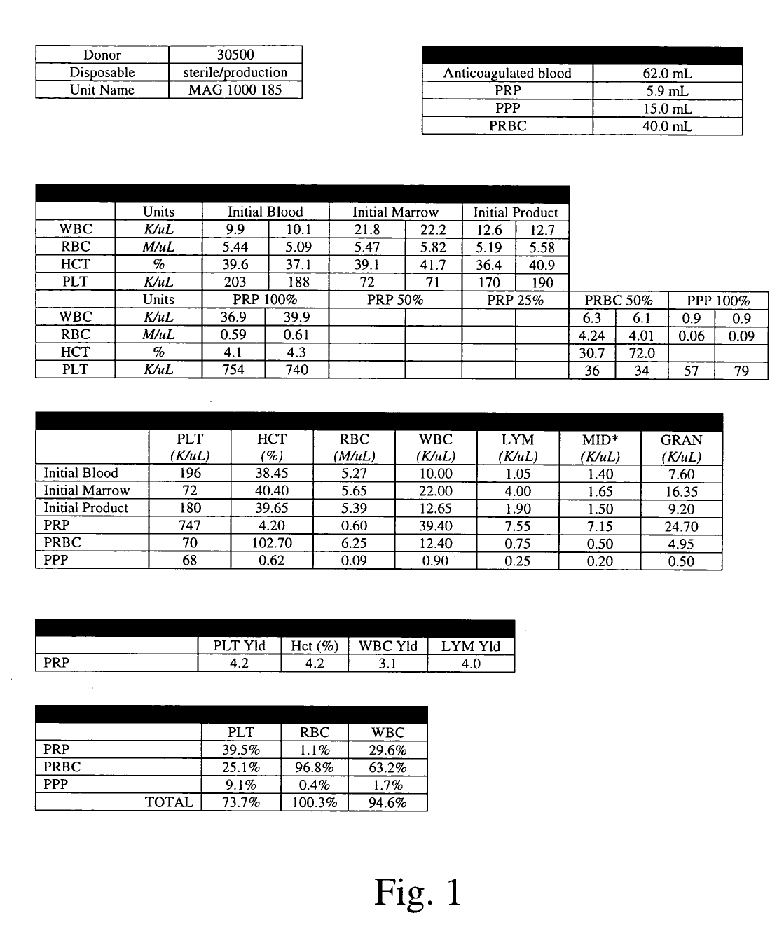

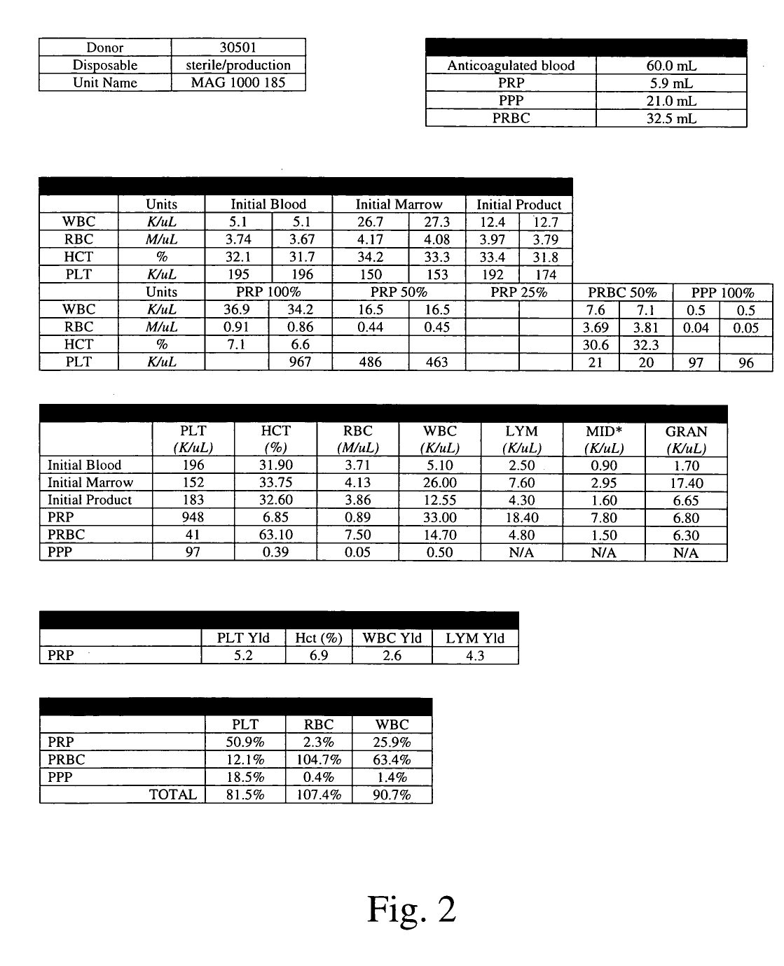

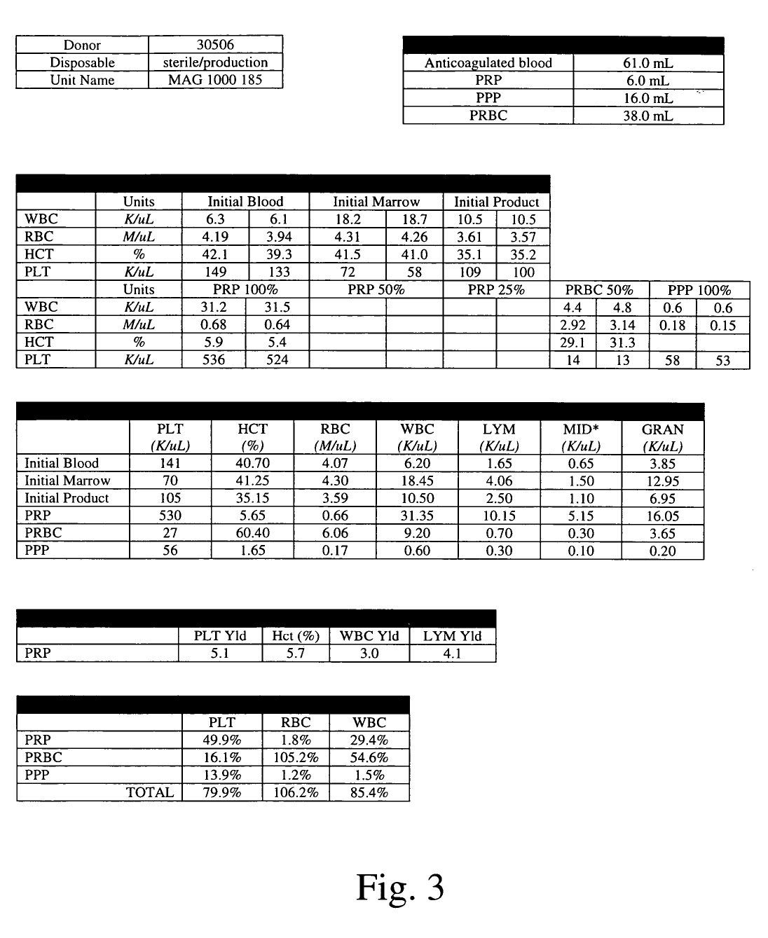

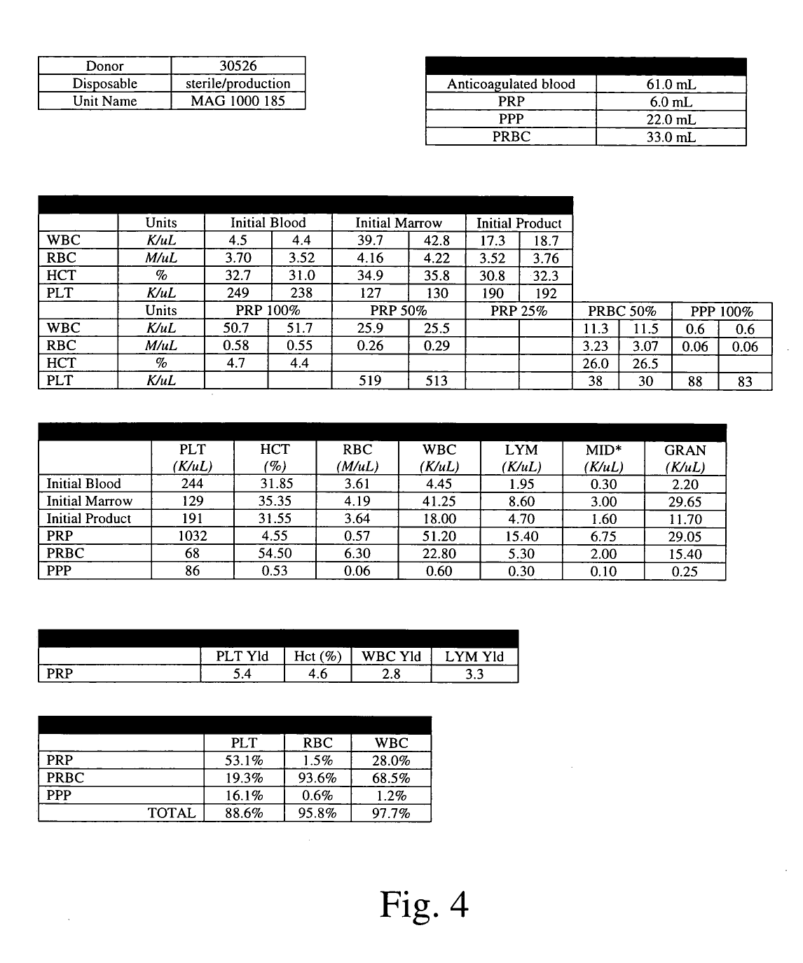

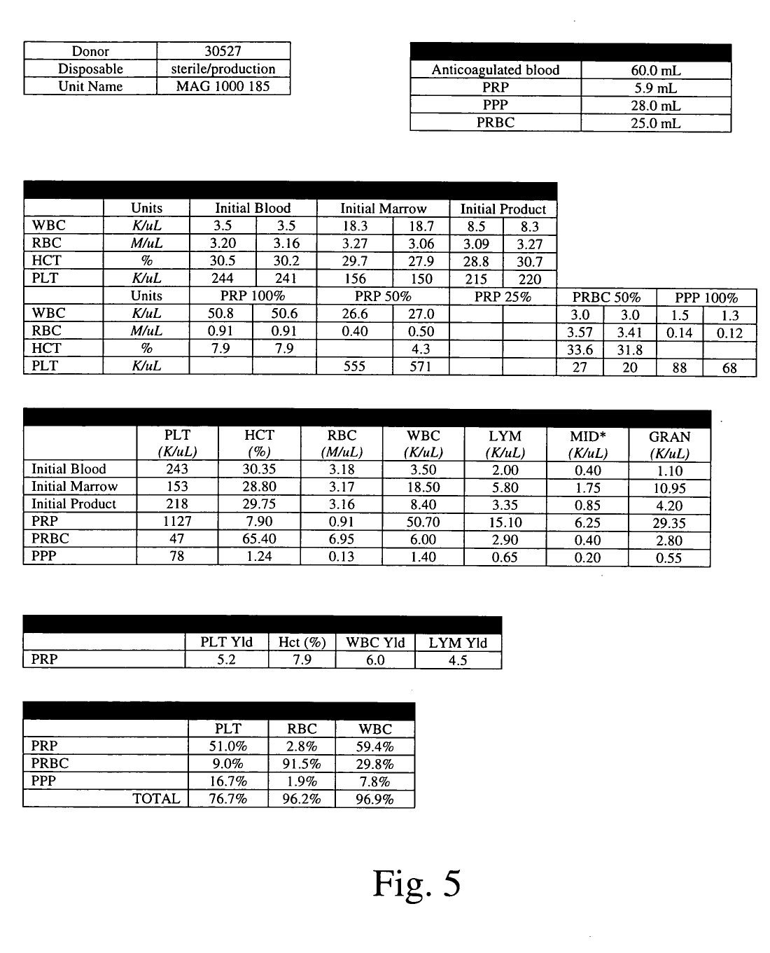

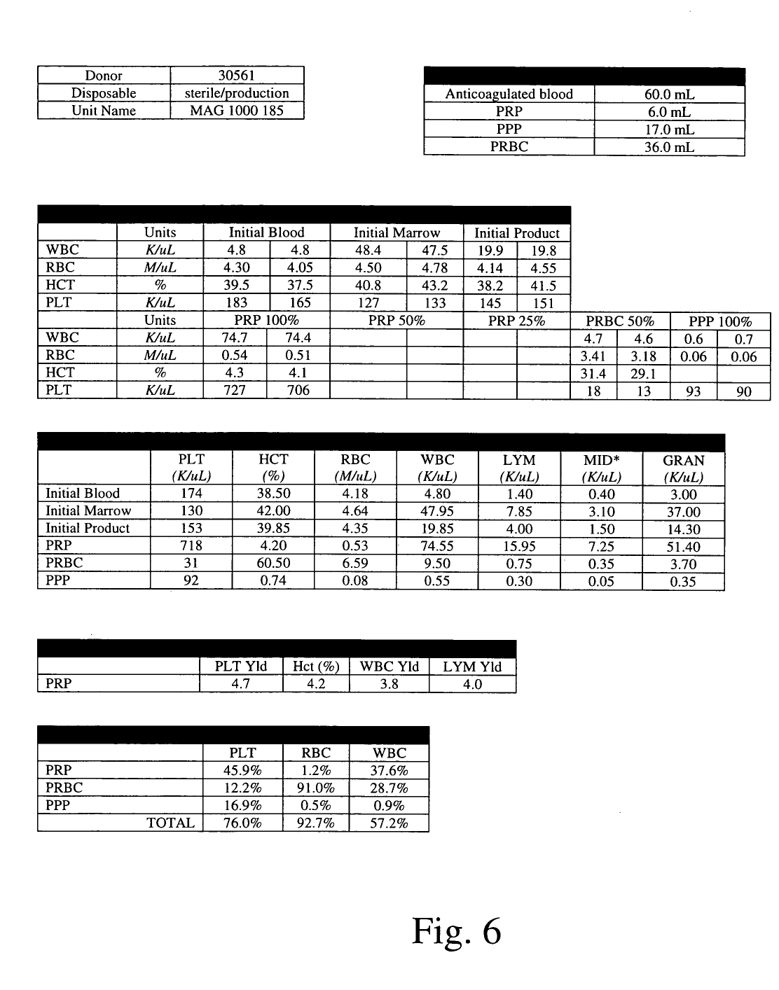

The testing results for each run are set forth in FIGS. 1-6 wherein FIG. 1 shows the testing results for donor number 30500, FIG. 2 shows the testing results for donor number 30501, FIG. 3 shows the testing results for donor number 30506, FIG. 4 shows the testing results for donor number 30526, FIG. 5 shows the testing results for donor number 30527, and FIG. 6 shows the testing results for donor number 30561. In FIGS. 1-6, the fraction rich in connective tissue growth promoting components is designated “PRP”. Other fractions of the biological sample are designated “PPP” for platelet poor plasma (i.e., the lowest density fraction), and “PRBC” for the red blood cell containing fraction (i.e., highest density fraction). Runs that were deemed unacceptable were excluded from the analysis. An acceptable separation run is defined as a run in which no untoward incidences are encountered. These untoward incidences include, but are not limited to: Failures due to operator error; Loss of ability to perform CBC counts in a reliable manner, and; Excessive platelet activation during venipuncture or transport which is manifested by excessive platelet clumping during or immediately after the separation process.

Equipment/Fixturing/Gauging Used

Magellan™ APS instrument, s/n MAG1000185 (equipped with software v. 2.3) Cell Dyn 1700 cell counter, Medtronic Equipment #133506.

Materials/Samples Used

Magellan™ Disposable kits, sterilized

Poietics Human Bone Marrow—Product Code 1M-125. Lot Numbers 030500, 030501, 030506, 030526, 030527, 030561. Poietics Normal.

Human Peripheral Blood—Product Code 1 W-406. Lot Numbers 030500, 030501, 030506, 030526, 030527, 03056.

Results and Data

The results of each run are summarized in the following table which shows the platelet yield and the % hematocrit by volume for the isolate rich in connective tissue growth components from each sample. Platelet Yield is the ratio of the platelet concentration in the isolate to that in the initial sample.

| Donor Lot | Platelet | Hematocrit |
|---|---|---|
| 030500 | 4.2 | 4.2 |
| 030501 | 5.2 | 6.9 |
| 030506 | 5.1 | 5.7 |
| 030526 | 5.4 | 4.6 |
| 030527 | 5.2 | 7.9 |
| 030561 | 4.7 | 4.2 |
| Average | 4.9 | 5.6 |
| Std Dev | 0.5 | 1.5 |

Conclusion

As can be seen from the above data, all six (6) separation runs conducted with the Magellan™ APS system had a concentration of platelets in the isolate rich in connective tissue growth promoting components (i.e., the PRP fraction) of greater than 4 times that of the original sample. In addition, all six (6) separation runs also resulted in an isolate rich in connective tissue growth promoting components (i.e., a PRP fraction) having a hematocrit (HCT) content of less than 12.5%.

Experimental 2

A connective tissue growth component rich fraction of a sample comprising blood and bone marrow has been isolated. Cells including mesenchymal stem cells in the isolate were then transfected with various doses of an adenoviral vector for hLMP-1 (i.e., AdVLMP). The cells were then implanted into rats using an athymic rat ectopic model.

While the foregoing specification teaches the principles of the present invention, with examples provided for the purpose of illustration, it will be appreciated by one skilled in the art from reading this disclosure that various changes in form and detail can be made without departing from the true scope of the invention.

All publications cited in the foregoing specification are hereby incorporated by reference in their entirety as if each had been individually incorporated by reference and fully set forth.

1. A method for obtaining a bone marrow fraction, comprising:
a) centrifuging a biological sample including bone marrow and whole blood to provide a separation of components of the sample based on density, said separation providing the following fractions in decreasing order of density:
(i) a fraction rich in blood cells;
(ii) a buffy coat fraction;
(iii) a platelet rich fraction; and
(iv) a platelet poor fraction; and
b) isolating the buffy coat fraction alone or in combination with all or part of the platelet rich fraction to form an isolate rich in connective tissue growth promoting components.

2. The method of claim 1, wherein:
said whole blood and bone marrow are from the same mammalian source.

3. The method of claim 2, wherein:
said mammalian source is a human.

4. The method of claim 1, also comprising combining said isolate rich in connective tissue growth promoting components with a carrier.

5. The method of claim 1, wherein said isolate rich in connective tissue growth promoting components comprises mesenchymal stem cells.

6. The method of claim 1, wherein said whole blood is peripheral blood.

7. The method of claim 1, also comprising:
harvesting said bone marrow from a patient during a surgical procedure.

8. The method of claim 7, wherein:
said centrifuging and isolating are conducted intraoperatively with said surgical procedure.

9. The method of claim 1, wherein said biological sample consists essentially of an anticoagulated mixture of bone marrow and whole blood.

10. The method of claim 1, wherein:
said biological sample centrifuged in the absence of any synthetic density gradient material.

11. The method of claim 1, wherein:
said centrifuging provides a platelet yield of at least about 2.

12. The method of claim 1, wherein:
said isolate rich in connective tissue growth components has a hematocrit content of less than 50% by volume.

13. The method of claim 1, wherein:
said isolate rich in connective tissue growth components has a platelet concentration greater than 4 times that of said biological sample, and a hematocrit content of less than about 12.5% by volume.

14. A method for treating a patient, comprising:
obtaining an isolate including a bone marrow fraction having components that promote connective tissue formation; and
implanting said isolate into a patient at a tissue defect site;
wherein said obtaining is performed intraoperatively with said implanting.

15. The method of claim 14, wherein:
said bone marrow fraction is autologous to the patient.

16. The method of claim 14, wherein:
said obtaining comprises centrifuging a biological sample including bone marrow.

17. The method of claim 15, wherein:
said obtaining includes harvesting bone marrow from the patient, centrifuging a biological sample comprising said bone marrow, and collecting said isolate.

18. The method of claim 17, wherein:
said biological sample also comprises whole blood.

19. The method of claim 18, wherein:
said whole blood is peripheral blood of the patient.

20. The method of claim 19, wherein:
said isolate has a platelet concentration greater than about 2 times that of said biological sample.

21. The method of claim 19, wherein:
said isolate has a hematocrit content of less than about 12.5% by volume.

22. The method of claim 14, wherein:
said implanting is at a bone defect site.

23. The method of claim 22, also comprising:
contacting said isolate with an osteogenic protein prior to, during, or after said implanting.

24. The method of claim 23, wherein:
said osteogenic protein is a bone morphogenic protein (BMP).

25. The method of claim 24, wherein said BMP is BMP-2 or BMP-7.

26. The method of claim 14, wherein:
said implanting is at a cartilage defect site.

27. The method of claim 17, wherein:
said biological sample is centrifuged in the absence of any synthetic density gradient material.

28. The method of claim 19, wherein:
said biological sample consists essentially of an anticoagulated mixture of bone marrow and peripheral blood of the patient.

29. The method of claim 14, also comprising:
combining said isolate with a carrier prior to said implanting.

30. The method of claim 29, wherein:
said carrier is resorbable.

31. The method of claim 30, wherein:
said carrier includes a dimensionally-stable porous matrix material.

32. The method of claim 31, wherein:
said porous matrix material comprises a natural or synthetic polymer.

33. The method of claim 31, wherein:
said porous matrix material comprises a member selected from the group consisting of collagen, gelatin, hyaluronic acid, carboxymethyl cellulose, and synthetic polymers.

34. The method of claim 33, wherein:
said porous matrix material comprises collagen.

35. The method of claim 32, wherein said carrier includes particulate mineral embedded in said porous matrix material.

36. The method of claim 30, wherein:
said carrier is a non-dimensionally-stable carrier.

37. The method of claim 36, wherein;
said carrier is a paste or putty.

38. The method of claim 36, wherein:
said carrier comprises an organic material selected from the group consisting of collagen, gelatin, hyaluronic acid, carboxymethyl cellulose, proteoglycans, glycosaminoglycans, and synthetic polymers.

39. The method of claim 38, wherein:
said carrier includes collagen or gelatin.

40. The method of claim 37, wherein:
said carrier includes particulate mineral in admixture with said organic material.

41. An implant composition, comprising:
an isolate material containing an uncultured bone marrow fraction including tissue growth promoting components, in combination with a biocompatible carrier.

42. The implant composition of claim 41, wherein:
said carrier provides a resorbable scaffold for tissue growth.

43. The implant composition of claim 41, wherein:
said isolate material has been obtained by centrifuging a biological sample including bone marrow and collecting said bone marrow fraction including tissue growth promoting components.

44. The implant composition of claim 41, wherein:
said isolate also comprises peripheral blood components.

45. The implant composition of claim 43, wherein:
said biological sample also includes peripheral blood.

46. The implant composition of claim 42, wherein:
said carrier comprises collagen.

47. The implant composition of claim 45, wherein:
said isolate material has a hematocrit content of less than about 12.5% by volume.

48. The implant composition of claim 41, wherein said tissue growth promoting components include mesenchymal stem cells.

49. A method for treating a patient, comprising:
providing a biological sample including tissue from a bone marrow source of the patient, said sample including tissue growth promoting components;
centrifuging the biological sample to separate the sample into fractions based on density, said fractions including a fraction rich in said tissue growth promoting components;
isolating said fraction rich in tissue growth promoting components;
implanting said fraction rich in tissue growth promoting components into the patient; and
wherein said centrifuging, and isolating are performed intraoperatively with said implanting.

50. The method of claim 49, wherein:
said centrifuging and isolating are effective to enrich said fraction in said growth promoting components relative to said biological sample.

52. The method of claim 49, wherein said growth promoting components include cells, and also comprising genetically modifying said cells.

53. The method of claim 49, wherein said biological sample also comprises whole blood.

54. The method of claim 53, wherein said whole blood is peripheral blood.

55. The method of claim 49, wherein:
said tissue is obtained from said bone marrow source by aspiration.

56. The method of claim 55, wherein:
said tissue is aspirated from an iliac crest of the patient.

57. The method of claim 49, wherein:
said biological sample is centrifuged in the absence of any synthetic density gradient material.

58. A method for obtaining an isolate material comprising tissue growth promoting components, comprising:
harvesting tissue from a bone marrow source of a mammal, said tissue including tissue growth promoting components;
forming an admixture by combining said tissue with whole blood of the mammal;
centrifuging a biological sample including said admixture so as to separate the biological sample into fractions based on density, said fractions including a fraction rich in said tissue growth promoting components; and
isolating said fraction rich in said tissue growth promoting components.

59. A method for obtaining a bone marrow fraction rich in connective tissue growth promoting components, the method comprising:
centrifuging a biological sample comprising bone marrow to separate components of the sample into fractions based on density, said fractions including a fraction rich in tissue promoting components; and
isolating said fraction rich in tissue growth promoting components.

60. A method for preparing a medical implant material for delivery, the method comprising:
(a) centrifuging a biological sample including a bone marrow material under conditions effective to separate a fraction including bone marrow components that promote tissue growth; and
(b) loading the fraction into a device for delivering the fraction to a patient without washing the fraction.

61. A method for preparing a medical implant material for delivery, the method comprising:
(a) centrifuging a biological sample including a bone marrow material under conditions effective to separate a fraction including bone marrow components that promote tissue growth; and
(b) loading the fraction into a device for delivering the fraction to a patient without culturing the fraction.

62. A medical combination comprising an isolated, uncultured bone marrow fraction enriched in components that promote tissue growth, in combination with a device for delivering the material to a patient.
